SEQUENCE LISTING (19 sequences)

SEQ ID NO: 1 - DNA, 1890 bp, Danio rubio
aagaatcacaaagcaatccgatgtaagttcggactacaaagccttgtggaatcatgtagacaccgactagactcgcttcaatcaaacgaacatccaccagaagcgctgggcgtgttcgtgttttgatgtgctcgagtcacaggttttgttcaacattcctcctggccgacgcgacgcggtgagcgagccgccgcacaaccgagtaatgctttcacttttccagcgagaagatggatgggaaatccaactacagccggctactcatcgcgctgctgatgattctgttttttggcggaattgtactgcaatacatatgttcaacatccgactggcagttactacacctggcatcattgtcctcgaggctggggagtcgcgcgcccggagatcgcttgaacggagccggtgcgggagatccgtacagctcggaggacggtgctttggttcgctttgtgcctcgttttaattttaccactaaagaccttagtcgcgcggtggatttccacattaagggggacgatgttattgtgttcctccacattcaaaaaaccggtgggaccacattcggccgtcacctggtccgcaacatccagctggagaggccgtgcgagtgccatgcgggccagaagaagtgtacctgctaccggccgggcaaacgcgacacctggctgttctcccgcttctccaccggctggagctgcggtctgcacgcggactggaccgagctcaccaactgcgtgccttccttcatgagcaaccgggagtcccaggagagacgcatgactcctagtaggaactactactacatcaccatcttgagggatccggtgtggcggtatctcagcgaatggaggcacgttcagcgtggagccacttggaaagcttctaaacacatgtgtgacggccgtttacccaccctgaccgagctgcccagctgttatcctggcgacgactggtccggctgctcgctggaggaattcatggtgtgcccttacaacctagccaacaacagacagacccgaatgctggccgacctcagcctggtgggctgctataacctcacggtaatgagcgagaaccagcggtgggccatgctactggaaagtgccaagcgcaacttgcgaaacatggccttttttggtctgaccgaatatcaacgtaagacgcagtacctgtttgaacacacgttccgtctttccttcatcgcaccttttacacagcttaacggcacccgtgctgcgagcgtagaagtggaaccggagacccaacgcagaattcgagagcttaaccaatgggacgtggagctatatgaatatgcacgggatcttttcctccagcgcttccagttcgccagacagcaggagcgcagggaggcccgtcagcgacgcatacaggagcggcgaaagctacgtgccaaggtgaagtcttggttgggggtgactggaaaagcggtttttaaacccaccaaggagccaccaatgacagagcagtcgcccgcttttgctgaagaaaaacaagcagatgctgaacaaacgctggagagcgagaccgaaggacaggtagaggagaactggctagaggaggatgacggtgaaatcatgttggactattcagaaaacgtagagcagtggcggtagcatttgggaagagggttgaacttaatactgaactagaaaatggtgattccatttgattactttatggtacctataatcctcgaagcccaaatcactgctgaatattagcaatatacgggttgtttattgtgggttgctttttatgtcacgtgcatttggtgaaatgcacatggaaatcttcatatgtatcagttattggctccagttctctgtcagatatcaatgaatgatcatctgcagcctgactcatctt

SEQ ID NO: 2 - protein, 553 aa, Danio rubio
MDGKSNYSRLLIALLMILFFGGIVLQYICSTSDWQLLHLASLSSRLGSRAPGDRLNGAGAGDPYSSEDGALVRFVPRFNFTTKDLSRAVDFHIKGDDVIVFLHIQKTGGTTFGRHLVRNIQLEKPCECHACQKKCTCYRPGKRDTWLFSRFSTGWSCGLHADWTELTNCVPSFMSNRESQERRMTPSRNYYYITILRDPVWRYLSEWRHVQRGATWKASKHMCDGRLPTLTELPSCYPGDDWSGCSLEEFMVCPYNLANNRQTRMLADLSLVGCYNLTVMSENQRWAMLLESAKRNLRNMAFFGLTEYQRKTQYLFEHTFRLSFIAPFTQLNGTRAASVEVEPETQRRIRELNQWDVELYEYARDLFLQRFQFARQQERREARQRRIQERRKLRAKVKSWLGVTGKAVFKPTKEPPMTEQSPAFAEEKQADAEQTLESETEGQVEENWLEEDDGEIMLDYSENVEQWRZHLGRGLNLILNZKMVIPFDYFMVPIILEAQITAEYZQYTGCLLWVAFYVTCIWZNAHGNLHMYQLLAPVLCQISMNDHLQPDSS

SEQ ID NO: 3 - DNA, 2564 bp, Homo sapiens
actgttccgcgggcaccggcagcgcagcgtctccgatagtaagtcgggctgccggccggctcattcccccagggtaactctgagcccccggctccgagctccctcgaggccgcctaccggcgtcgggaacatggatgagaaatccaacaagctgctgctagctttggtgatgctcttcctatttgccgtgatcgtcctccaatacgtgtgccccggcacagaatgccagctcctccgcctgcaggcgttcagctccccggtgccggacccgtaccgctcggaggatgagagctccgccaggttcgtgccccgctacaatttcacccgcggcgacctcctgcgcaaggtagacttcgacatcaagggcgatgacctgatcgtgttcctgcacatccagaagaccgggggcaccactttcggccgccacttggtgcgtaacatccagctggagcagccgtgcgagtgccgcgtgggtcagaagaaatgcacttgccaccggccgggtaagcgggaaacctggctcttctccaggttctccacgggctggagctgcgggttgcacgccgactggaccgagctcaccagctgtgtgccctccgtggtggacggcaagcgcgacgccaggctgagaccgtccaggaacttccactacatcaccatcctccgagacccagtgtcccggtacttgagtgagtggaggcatgtccagagaggggcaacatggaaagcatccctgcatgtctgcgatggaaggcctccaacctccgaagagctgcccagctgctacactggcgatgactggtctggctgccccctcaaagagtttatggactgtccctacaatctagccaacaaccgccaggtgcgcatgctctccgacctgaccctggtaggctgctacaacctctctgtcatgcctgaaaagcaaagaaacaaggtccttctggaaagtgccaagtcaaatctgaagcacatggcgttcttcggcctcactgagtttcagcggaagacccaatatctgtttgagaaaaccttcaacatgaactttatttcgccatttacccagtataataccactagggcctctagtgtagagatcaatgaggaaattcaaaagcgtattgagggactgaattttctggatatggagttgtacagctatgccaaagacctttttttgcagaggtaccagtttatgaggcagaaagagcatcaggaggccaggcgaaagcgtcaggaacaacgcaaatttctgaagggaaggctccttcagacccatttccagagccagggtcagggccagagccagaatccgaatcagaatcagagtcagaacccaaatccgaatgccaatcagaacctgactcagaatctgatgcagaatctgactcagagtttgagccagaaggagaaccgggaaagcccgaagcagaactcaggcaaggagcagaatgataacaccagcaatggcaccaacgactacataggcagtgtagagaaatggcgttaaatggctcaaaaaggcctgtacatacttctcccaaagcgccactgaaaagatggcatagcttaaaagatgaaagtgtccaaacacatcctgcttccttcattggggaagttttaaaaaaaagtttagatgttgcctttacagttgcctttcaattcagtgttatactgtgtgtaggtaaaacaaatctcaatatggaattaaattgtctttttggggttggactaaatatgaaatccgaaagccaaaccagactcaccagaaattgctgtttagatattttaagaagttcttaaattagttatggagacaaagtgaaaacataaaatgtgaccatttaacttatggctaagaaatggactttaaattattcatgatacactgttaaaacccaatcttggaatcaaatattttttccaggggtgagaataagtataaacataaagcaactaaaatgaaacataaaaccttttattttcttctgattttaacaaggaatctatttaaatagaataacaactgatggtgaatcttaccgagctgtagaaaataaaaaattcctctccaaacatgggtagttttatgtcaaaatattggcttttcaagaacaggactcatatcttgatatttaagagatgtttaaaattttaaactttttctaccttctactgtttaaaggttttacacagggtgtatctcacattaaacaaaacacctttttttcaattttctttagttttaattgaaaatgtttgcttttaaaactgataggtattgttggaaagcaggatgaagcctgagccagtggaaaagcttgttacagaaaaaacattttgtgttattgctgtggtgtgcatgatttgcaaagattaagtgcattttctctgtctatactgattattgtatatagaggatgttataaatatacatatacatttttgccattatgtaaatcccatgatttcaactgtaaacatctgtccattggtgtagctttacaaaccattcactgattttgtgtaatttaacaatagatatgaaataaagtttaaattac

SEQ ID NO: 4 - protein, 460 aa, Homo sapiens
MDEKSNKLLLALVMLFLFAVIVLQYVCPGTECQLLRLQAFSSPVPDPYRSEDESSARFVPRYNFTRGDLLRKVDFDIKGDDLIVFLHIQKTGGTTFGRHLVRNIQLEQPCECRVGQKKCTCHRPGKRETWLFSRFSTGWSCGLHADWTELTSCVPSVVDGKRDARLRPSRNFHYITILRDPVSRYLSEWRHVQRGATWKASLHVCDGRPPTSEELPSCYTGDDWSGCPLKEFMDCPYNLANNRQVRMLSDLTLVGCYNLSVMPEKQRNKVLLESAKSNLKHMAFFGLTEFQRKTQYLFEKTFNMNFISPFTQYNTTRASSVEINEEIQKRIEGLNFLDMELYSYAKDLFLQRYQFMRQKEHQEARRKRQEQRKFLKGRLLQTHFQSQGQGQSQNPNQNQSQNPNPNANQNLTQNLMQNLTQSLSQKENRESPKQNSGKEQNDNTSNGTNDYIGSVEKWRZ

SEQ ID NO: 5 - DNA, 21 bp, Artificial Sequence
  Oligonucleotide
tcctgtgtgaaattgttatcc

SEQ ID NO: 6 - DNA, 19 bp, Artificial Sequence
  Oligonucleotide
ctcctcagtacacatatgg

SEQ ID NO: 7 - DNA, 19 bp, Artificial Sequence
  Oligonucleotide
gctgatgggacagtggatt

SEQ ID NO: 8 - DNA, 19 bp, Artificial Sequence
  Oligonucleotide
gaagaagtgtacctgctac

SEQ ID NO: 9 - DNA, 19 bp, Artificial Sequence
  Oligonucleotide
cacggtaatgagccgagaa

SEQ ID NO: 10 - DNA, 18 bp, Artificial Sequence
  Oligonucleotide
ccagctttgttcagcatc

SEQ ID NO: 11 - DNA, 18 bp, Artificial Sequence
  Oligonucleotide
ctggctgttctcccgctt

SEQ ID NO: 12 - DNA, 25 bp, Artificial Sequence
  Oligonucleotide
gatttcccatccatcttctcgctgg

SEQ ID NO: 13 - DNA, 25 bp, Artificial Sequence
  Oligonucleotide
agtgaaagcattactcggttgtgcg

SEQ ID NO: 14 - DNA, 25 bp, Artificial Sequence
  Oligonucleotide
agtcaatgcattagtcggttctgcg

SEQ ID NO: 15 - DNA, 25 bp, Artificial Sequence
  Oligonucleotide
gtatcaaataaacaaccaagttcat

SEQ ID NO: 16 - DNA, 33 bp, Artificial Sequence
  Oligonucleotide
gaagatctcaccatggatgagaaatccaacaag

SEQ ID NO: 17 - DNA, 29 bp, Artificial Sequence
  Oligonucleotide
gaagatctttaacgccatttctctacact

SEQ ID NO: 18 - protein, 507 aa, Mus musculus
MDEKSNKLLLALVMLFLFAVIVLQYVCPGTECQLLRLQAFSSPVPDPYRSEDESSARFVPRYNFSRGDLLRKVDFDIKGDDLIVFLHIQKTGGTTFGRHLVRNIQLEQPCECRVGQKKCTCHRPGKRETWLFSRFSTGWSCGLHADWTELTSCVPAVVDGKRDARLRPSRWRIFQILDGTSKDRWGSSNFNSGANSPSSTKPPRSTSKSGKNFHYITILRDPVSRYLSEWRHVQRGATWKASLHVCDGRPPTSEELPSCYTGDDWSGCPLKEFMDCPYNLANNRQVRMLSDLTLVGCYNLSVMPEKQRNKVLLESAKSNLKHMAFFGLTEFQRKTQYLFEKTFNMNFISPFTQYNTTRASSVEINEEIQKRIEGLNFLDMELYSYAKDLFLQRYQFMRQKEHQDARRKRQEQRKFLKGRFLQTHFQSQSQGQSQSQSPGQNLSQNPNPNPNQNLTQNLSHNLTPSSNPNSTQRENRGSQKQGSGQGQGDSGTSNGTNDYIGSVETWR

SEQ ID NO: 19 - protein, 469 aa, Mus musculus
MDERFNKWLLTPVLTFLFVVIMYQYVSPSCTSSCTNFGEQLRSGEARPPAVPSPARRAQAPLDEWERRPQLPPPPRGPPEGSRGVAAPEDEDEDPGDPEEEEEEEEEEPDPEAPENGSLPRFVPRFNFTLKDLTRFVDFNIKGRDVIVFLHIQKTGGTTFGRHLVKNIRLEQPCSCKAGQKKCTCHRPGKKETWLFSRFSTGWSCGLHADWTELTNCVPAIMEKKDCPRNHSHTRNFYYITMLRDPVSRYLSEWKHVQRGATWKTSLHMCDGRSPTPDELPTCYPGDDWSGVSLREFMDCSYNLANNRQVRMLADLSLVGCYNLTFMNESERNTILLQSAKNNLKNMAFFGLTEFQRKTQFLFERTFNLKFISPFTQFNITRASNVDINDGARQHIEELNFLDMQLYEYAKDLFQQRYHHTKQLEHQRDRQKRREERRLQREHRAHRWPKEDRAMEGTVTEDYNSQVVR

(19) United States
(12) Patent Application Publication
(10) Pub. No.: US 2004/0241683 A1
(43) Pub. Date: Dec. 2, 2004

(54) Hsst and angiogenesis

(76) Inventors: Stephen C Ekker, St Paul, MN (US); Aidas Nasevicius, St Paul, MN (US); Eleanor Y Chen, Minneapolis, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/483,038

(22) PCT Filed: Jul. 3, 2002
(86) PCT No.: PCT/US02/21133

Related U.S. Application Data
(60) Provisional application No. 60/303,764, filed on Jul. 6, 2001.

(51) Int. Cl. C12Q 1/68; A01K 67/027; C07D 473/02; C07D 413/14; C07K 16/18

(52) U.S. Cl. 435/6; 435/69.1; 435/320.1; 435/326; 530/388.1; 800/20; 544/81

(57) ABSTRACT

The invention provides methods and materials related to modulating angiogenesis in an animal. The invention provides polynucleotides and modified polynucleotides such as morpholino-modified polynucleotides for modulating angiogenesis, as well as cells and embryos containing these polynucleotides. The invention also provides methods for identifying HSST- and angiogenesis-modulating agents.

### BACKGROUND

1. Technical Field

The invention relates to methods and materials involved in modulating angiogenesis in an animal.

2. Background Information

Heparan sulfate proteoglycans are present ubiquitously on the cell surface and in extracellular matrix. They interact with a variety of proteins such as heparin binding growth factors and components of extracellular matrices to modulate many cellular processes including cell adhesion, proliferation, and differentiation. Heparan sulfate proteoglycans also have been shown to participate in a wide-range of physiological phenomena including blood coagulation, inflammation, microbial invasion, and tumor metastasis. Interactions between heparan sulfate proteoglycans and ligands seem to involve recognition of specific domains on heparan sulfate chains by ligands leading to subsequent binding.

To generate specific domains in a heparan sulfate chain, enzymes catalyzing epimerization and sulfation reactions are involved. Sulfation, especially, is an important modification as specific domains of sulfation on heparan sulfate chains are recognized and bound by specific ligands.

To date, several enzymes involved in sulfation of specific residues of heparan sulfate chains have been identified. It has been shown that targeted disruption of a mouse gene encoding heparan sulfate 2-O-sulfotransferase (HS2ST) causes severe defects in kidney development (Bullock et al. (1998) Genes and Development 12:1894-1906). On the other hand, three homologues of heparan sulfate 6-O-sulfotransferase (HS6ST) have been identified in mouse, and each has its own substrate specificity (Habuchi et al. (2000) J of Biol Chem 275:2859-2868). A human HS6ST cDNA also has been isolated from Chinese hamster ovary cells and a human fetal brain cDNA library (see Habuchi et al. (1998) J Biol Chem 273:9208-9213.) This human HS6ST is most similar to the mouse homologue HS6ST-1. The human HS6ST has been shown in vitro to be a secreted protein. To date, however, the biological significance of HS6ST in vertebrates is not known.

### SUMMARY

The invention provides methods and materials related to modulating angiogenesis in an animal. The invention is based on the discovery that a zebrafish HSST homologue is involved in angiogenesis. Therefore, the invention provides methods and materials for modulating angiogenesis by modulating the activity or expression of an HSST polypeptide. For example, the invention provides modified polynucleotides such as morpholino-modified polynucleotides that can be used to decrease expression from nucleic acids encoding HSST polypeptides. The invention also provides assays that can be used to identify HSST modulators that decrease or increase the biological effects of HSST by decreasing or increasing HSST expression or enzymatic activity. In addition, HSST modulators can be used to manage or treat disease conditions associated with angiogenesis.

The invention provides a morpholino-modified HSST polynucleotide that is complementary to a nucleic acid molecule that encodes an HSST polypeptide. The morpholino-modified HSST polynucleotide is effective to decrease expression from the nucleic acid molecule encoding an HSST polypeptide. In one embodiment, the morpholino-modified HSST polynucleotide is effective to decrease expression of the zebrafish EC1 polypeptide (SEQ ID NO: 2). In another embodiment, the morpholino-modified HSST polynucleotide is effective to decrease expression of the human AK polypeptide (SEQ ID NO: 4).

In another embodiment, the invention provides a cell comprising a morpholino-modified HSST polynucleotide that is complementary to a nucleic acid molecule that encodes an HSST polypeptide. The invention also provides a teleost embryo that has a morpholino-modified HSST polynucleotide that is complementary to a nucleic acid molecule that encodes an HSST polypeptide. The morpholino-modified HSST polynucleotide is effective to decrease expression from a nucleic acid molecule encoding an HSST polypeptide. The decreased expression from a nucleic acid molecule encoding an HSST polypeptide results in an alteration of angiogenesis in the embryo. The teleost embryo can be a zebrafish embryo, a stickleback embryo, a medaka embryo, and a puffer fish embryo.

In another embodiment, the invention provides an expression vector having an expression control sequence and a coding sequence. The expression control sequence and the coding sequence are operably linked such that the expression control sequence directs production of a polynucleotide from the coding sequence. The polynucleotide can be complementary to the zebrafish ec1 nucleotide sequence (SEQ ID NO: 1) or to the human ak nucleotide sequence (SEQ ID NO: 3).

In another embodiment, the invention provides a purified polypeptide having the zebrafish EC1 polypeptide sequence (SEQ ID NO: 2). The invention also provides a purified antibody that binds specifically to the zebrafish EC 1 polypeptide as well as a purified antibody that binds specifically to the human AK polypeptide (SEQ ID NO: 2 and SEQ ID NO: 4, respectively).

In another embodiment, the invention provides a method of making an antibody that includes immunizing a non-human animal with the EC1 or human AK polypeptide, or an immunogenic fragment of the zebrafish EC1 or human AK polypeptide (SEQ ID NO: 2 and SEQ ID NO: 4, respectively). The invention also provides a method of making a monoclonal antibody that involves (1) providing a hybridoma cell that produces a monoclonal antibody specific for the zebrafish EC1 polypeptide or human AK polypeptide, and culturing the hybridoma cell under conditions that permit production of the monoclonal antibody.

In another embodiment, the invention provides a method of identifying an HSST-modulating agent. The method involves (1) contacting a test agent with a cell that produces an HSST polypeptide, (2) detecting the amount or activity level of the HSST polypeptide after step (1), and (3) identifying the test agent as an HSST-modulating agent if the amount or activity level of the HSST polypeptide is increased or decreased relative to a control cell.

The invention also provides another method of identifying an HSST-modulating agent involving (1) contacting a test agent with a purified or partially purified polypeptide preparation having enzymatically active HSST polypeptide, (2) detecting the activity of the HSST polypeptide after step (1), and identifying the test agent as an HSST-modulating agent if the activity of the HSST polypeptide is increased or decreased compared to the activity in a control purified or partially purified HSST polypeptide preparation.

In another embodiment, the invention provides a method of identifying an angiogenesis-modulating agent. This method involves contacting an animal with an HSST-modulating agent and monitoring the animal that has been contacted with the HSST-modulating agent for any alteration in angiogenesis. The HSST-modulating agent is identified as an angiogenesis-modulating agent if any alteration in angiogenesis is detected.

In another embodiment, the invention provides a method of making an angiogenesis-modulating agent. This method involves contacting an animal with an HSST-modulating agent and monitoring the animal that has been contacted with the candidate agent for any alteration in angiogenesis. The HSST-modulating agent is identified as an angiogenesis-modulating agent if any alteration in angiogenesis is detected. The angiogenesis-modulating agent is then produced or manufactured for commercial purposes.

In another embodiment, the invention provides a method of modulating angiogenesis in an animal such as a zebrafish. The method involves introducing an angiogenesis-modulating agent into the animal. The angiogenesis-modulating agent can be a polynucleotide or an antibody. The polynucleotide can be a morpholino-modified polynucleotide. In addition, the polynucleotide can be encoded by an expression vector.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is the nucleotide sequence of the zebrafish ec1 gene. (SEQ ID NO: 1)

FIG. 2 is the zebrafish EC1 polypeptide sequence. (SEQ ID NO: 2)

FIG. 3 shows the percentages of embryos exhibiting decreased or no blood vessel formation subsequent to injection with either of the two ec1-MOs or the ec1-MO (Δ4) control.

FIG. 4 is a bar graph comparing the percentages of embryos exhibiting a strong or a weak VEGF morphant phenotype when injected with ec1-MO alone (HSST, 3 ng), vegf-MO alone (VEGF, 3ng), ec1-MO and veg-MO (H+V, 3ng each), ec1-MO at 6 ng (HSST, 6ng), or vegf-MO at 6ng (VEGF, 6ng).

FIG. 5 is a bar graph comparing the percentages of embryos exhibiting axial vessel deficiency when injected with ec1-MO alone, vegf-MO alone, both ec1-MO and vegf-MO, or ec1-MO and a control-MO.

FIG. 6 is the nucleotide sequence of the human ak gene (GenBank Accession AK027720). (SEQ ID NO: 3)

FIG. 7 is the human AK polypeptide sequence. (SEQ ID NO: 4)

FIG. 8 is an alignment of human, mouse, and zebrafish HSST polypeptide sequences.

### DETAILED DESCRIPTION

The invention provides methods and materials related to modulating angiogenesis in an animal. The invention is based on the discovery that a zebrafish HSST homologue is involved in angiogenesis. Therefore, the invention provides methods and materials for modulating angiogenesis by modulating the activity or expression of an HSST polypeptide. For example, the invention provides modified polynucleotides such as morpholino-modified polynucleotides that can be used to decrease expression from nucleic acids encoding HSST polypeptides. The invention also provides assays that can be used to identify HSST modulators that decrease or increase the biological effects of HSST by decreasing or increasing HSST expression or enzymatic activity. In addition, HSST modulators can be used to manage or treat disease conditions associated with angiogenesis.

1. Modified Polynucleotides

A polynucleotide is a polymer of three or more nucleotide subunits linked by phosphodiester bonds. A modified polynucleotide can be formed by replacing all or portions of the five-carbon sugar-phosphate backbone of a polynucleotide with alternative functional groups. Examples of modified polynucleotides include: morpholino-modified polynucleotides in which the bases are linked by a morpholino-phosphorodiamidate backbone (U.S. Pat. Nos. 5,142,047 and 5,185,444); polynucleotides in which the bases are linked by a polyvinyl backbone (Pitha et al. (1970) Biochem Biophys Acta 204:39 and Pitha et al. (1970) Biopolymers 9: 965); peptide nucleic acids (PNAs) in which the bases are linked by amide bonds formed by pseudopeptide 2-aminoethyl-glycine groups; polynucleotides in which the nucleoside subunits are linked by methylphosphonate groups (Miller et al. (1979) Biochem 18: 5134; Miller et al. (1980) J Biol Chem 255: 6959); polynucleotides in which the phosphate residues linking nucleoside subunits are replaced by phosphoroamidate groups (Froehler et al. (1988) Nucleic Acids Res 156: 4831); and phosphorothioated DNAs, polynucleotides containing sugar moieties that have 2′ O-methyl groups (Cook (1998) Antisense Medicinal Chemistry, Chapter 2, Antisense Research and Application, Springer, New York pages 51-101). Modified polynucleotides can be obtained commercially, produced using commercially available monomeric subunits, or synthesized using known methods. (See Braasch and Corey (2001) Chemistry and Biology pages 1-7)

Typically, modified polynucleotides such as morpholino-modified polynucleotides are single stranded and can be various lengths such as 8 to more than 112 bases in length. Modified polynucleotides such as morpholino-modified polynucleotides can be 12 to 72 bases in length. For example, modified polynucleotides such as morpholino-modified polynucleotides can be 15 to 45 bases in length. Ideally, morpholino-modified polynucleotides are 18-30 bases in length.

In addition, a modified polynucleotide such as a morpholino-modified polynucleotide can be sequence-specific. A modified polynucleotide that is sequence-specific is one that can anneal in a sequence-specific manner with a target polynucleotide such that expression from the target polynucleotide is altered, e.g. expression is decreased. As used herein, the term “expression,” with respect to expression from a target polynucleotide, refers to production of a functional RNA molecule from a DNA molecule, or production of a functional polypeptide from an mRNA molecule.

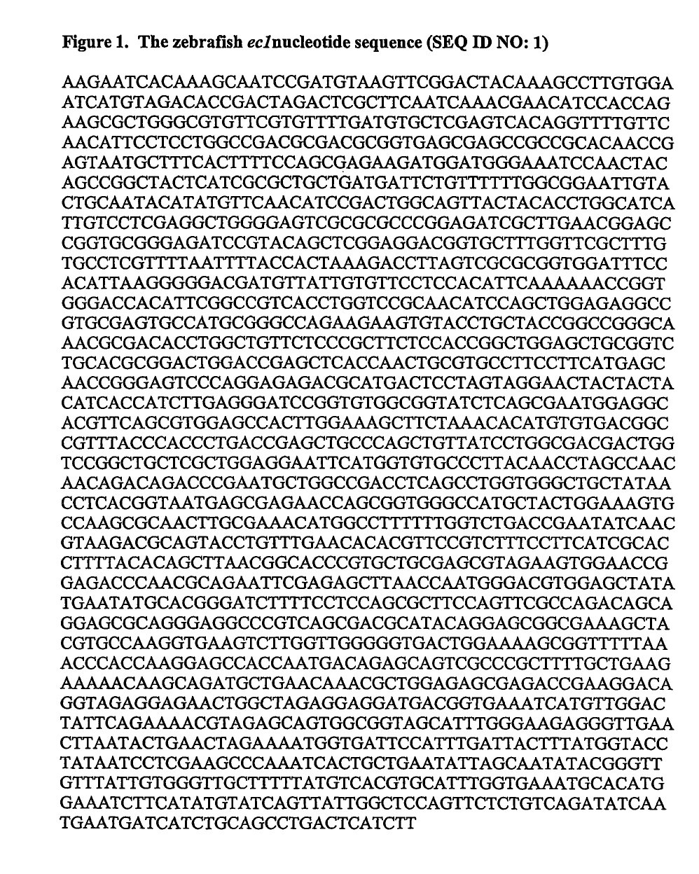

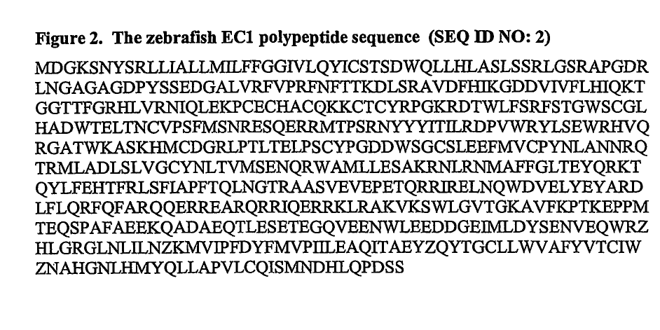

To be sequence-specific, a modified polynucleotide can have a sequence that is 100% complementary with the sequence of a portion of the target polynucleotide, i.e., all the nucleotides in the modified polynucleotide are able to anneal through hydrogen bonding, for example, with the nucleotides in the corresponding portion of the target polynucleotide according to known Watson-Crick type base pairing rules, e.g., adenine (A) pairs with thymine (T) and guanine (G) pairs with cytosine (C) (see, DNA in Molecular Cell Biology, Darnell et al. (1990) Scientific American Books. 2^{nd} Edition, pages 68-74). Examples of modified polynucleotides that are 100% complementary to their target polynucleotides include the morpholino-modified HSST polynucleotides ec1-MO #1 (SEQ ID NO: 12) and ecl-MO #2 (SEQ ID NO: 13); see Example 5. These morpholino-modified polynucleotides are 100% complementary to a portion of the zebrafish HSST sequence shown in FIG. 1 (SEQ ID NO: 1).

In addition, a modified polynucleotide can be considered sequence-specific even though it has a sequence that is not 100% complementary to the corresponding region in the target polynucleotide. When not 100% complementary, a polynucleotide can be sequence-specific provided the polynucleotide has a sufficient number of complementary nucleotides such that the polynucleotide can anneal in a sequence-specific manner to the corresponding region in the target polynucleotide to achieve sequence-specific alteration of expression from the target polynucleotide under particular conditions, e.g. intracellular conditions. As used herein, the term “complementary” refers to a polynucleotide sequence that is 100% complementary, as well as a polynucleotide sequence that is less than 100% complementary, to a portion of a target polynucleotide, provided sequence-specific alteration in expression from the target polynucleotide can be achieved under intracellular conditions. As used herein, intracellular conditions refer to conditions typical of the interior of a living cell existing in vitro or in vivo.

To determine whether a modified polynucleotide is complementary with a selected target polynucleotide, i.e., whether a modified polynucleotide has a sufficient proportion of complementary nucleotides with the target to mediate sequence-specific alteration in expression from the target, the modified polynucleotide can be compared to a negative control polynucleotide and a positive control polynucleotide in an expression study. A negative control polynucleotide is a similarly modified polynucleotide in which less than half of the nucleotides are able to pair with the selected portion of the target polynucleotide. The “mismatched” nucleotides in the negative control can be evenly distributed through the length of the negative control polynucleotide; see, for example, the sequence of ec1-MO (Δ4) (SEQ ID NO: 14). The negative control polynucleotide does not anneal with the target polynucleotide and does not alter expression from the target polynucleotide. A positive control polynucleotide is a similarly modified polynucleotide that is 100% complementary with the selected portion of the target polynucleotide and anneals with the target polynucleotide to alter expression from the target polynucleotide. Effects of the control polynucleotides and the modified polynucleotide in question on expression from the target polynucleotide can be compared. A modified polynucleotide having some mismatched nucleotides is considered complementary to a target polynucleotide if the modified polynucleotide is capable of altering expression from the target polynucleotide in a statistically significant amount when compared with the negative control polynucleotide. Expression studies could be performed in vivo or in vitro (e.g. in vitro transcription and translation). Methods for assessing expression are known in the art and include, without limitation, RNA hybridization assays or polypeptide hybridization assays.

A modified polynucleotide of about 25 nucleotides can have as many as three non-complementary nucleotides distributed throughout the modified polynucleotide and still be able to anneal with the target polynucleotide and mediate sequence-specific alteration in expression from the target polynucleotide. On the other hand, a polynucleotide of about 25 nucleotides in length having approximately 50% A and T nucleotides and 16% or more mismatches with a target polynucleotide is unable to mediate sequence-specific alteration in expression from the target polynucleotide. A polynucleotide having a proportion (e.g. 20%, 25%, 30%, 50% or 100%) of mismatched nucleotides with a target polynucleotide such that the polynucleotide does not anneal with the target under intracellular conditions is considered non-complementary. An example of a modified polynucleotide that is considered non-complementary with the selected nucleic acid encoding the zebrafish HSST homologue EC1 (SEQ ID NO: 1) is the ec1-MO (Δ4) polynucleotide (SEQ ID NO: 14); see Example 5.

The target polynucleotide can be any non-recombinant or recombinant nucleic acid. As used herein, the term “nucleic acid” includes, without limitation, cellular DNA such as genomic DNA; cellular RNA such as mRNA; eukaryotic or prokaryotic vectors such as plasmid DNA, cosmid DNA, phage DNA (e.g. λ DNA or M13 DNA), or viral DNA (e.g. DNA of a retrovirus, adenovirus, or herpes virus); other recombinant nucleic acids such as cDNA or DNA produced by restriction enzyme digestion or polymerase chain reaction (PCR); and synthetic polynucleotides such as DNA produced by chemical synthesis. As used herein, the term “vector” refers to a single or double stranded nucleic acid that does not rely on the genomic origin of replication to replicate in a cell. A vector can have expression control sequences (e.g. an expression vector) as well as coding sequences, both of which are discussed further below. A nucleic acid can exist (1) as a separate molecule such as a cDNA, a genomic DNA fragment, or a single-stranded polynucleotide; or (2) incorporated into a vector or the genomic DNA of a prokaryote or eukaryote. A recombinant or synthetic nucleic acid molecule can be part of a hybrid or fusion nucleic acid.

Thus, the target polynucleotide, i.e., the selected nucleic acid, can be, without limitation, cellular mRNA or genomic DNA, and the modified polynucleotide can have a sequence complementary to the mRNA molecule or to the DNA strand from which an mRNA is transcribed (the antisense strand).

Furthermore, a modified polynucleotide can be complementary to the coding region of an mRNA molecule or the region corresponding to the coding region on the antisense DNA strand. As used herein, the term “coding sequence” refers to the portion of a selected nucleic acid (DNA or RNA) that encodes a polypeptide. (A coding sequence can also include a nucleic acid that encodes a polynucleotide such as an RNA molecule that may or may not have an open reading frame from which a polypeptide can be translated.) A modified polynucleotide also can be complementary to the non-coding region of a selected nucleic acid molecule. A non-coding region, for example, can be a region upstream of a transcriptional start point or a region downstream of a transcriptional end-point in a DNA molecule. A non-coding region also can be a region upstream of the translational start codon or downstream of the stop codon in an mRNA molecule. A modified polynucleotide also can be complementary to both coding and non-coding regions of a selected nucleic acid molecule. A modified polynucleotide that is complementary to both coding and non-coding regions of a selected nucleic acid, for example, is one that is complementary to a region that includes a portion of the 5′ untranslated region leading up to the start codon, the start codon, and coding sequences immediately following the start codon of a selected mRNA. The sequence of the modified polynucleotide is selected to achieve maximum alteration of expression from the selected nucleic acid molecule with which it anneals.

With reference to nucleic acid, the term “isolated” as used herein refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prolcaryote or eukaryote. In addition, an isolated nucleic acid can include a recombinant DNA molecule that is part of a hybrid or fusion nucleic acid sequence.

The term “isolated” as used herein also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered isolated. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

2. Inhibition of Expression by Modified Polynucleotides

Modified polynucleotides such as morpholino-modified polynucleotides can be used to decrease expression from a selected nucleic acid molecule of known sequence. Expression from a nucleic acid molecule can be decreased by interfering with any process necessary for (1) RNA transcription, (2) RNA processing, (3) RNA transport across the nuclear membrane, (4) RNA translation, or (5) RNA degradation.

Expression from a selected nucleic acid molecule such as a DNA molecule can be decreased by interfering with processes necessary for formation of a functional RNA molecule or transport of the RNA into the cytoplasm. Processes necessary for formation of a functional RNA molecule include RNA polymerase binding to promoter regions, binding of transcriptional activator to its recognition sequence, transcription, or RNA processing. A modified polynucleotide that anneals to DNA and interferes with processes necessary for formation of a functional RNA molecule has a sequence that is complementary to the antisense DNA strand from which mRNA is transcribed, and is referred to as an “antigene” molecule.

Expression from a selected nucleic acid molecule such as an mRNA molecule can be decreased by interfering with any process necessary for translation of an mRNA into a functional polypeptide. Expression from an mRNA molecule, for example, can be decreased by interfering with ribosome binding to the ribosome-binding site, interfering with initiation of translation, interfering with the translation process, or interfering with proper termination of translation. A modified polynucleotide that anneals to a portion of an mRNA molecule and interferes with translation has a sequence that is complementary to that portion of the mRNA molecule and is referred to as an antisense polynucleotide. Antisense polynucleotides also can decrease expression by inducing the cellular nuclease system that degrades cognate mRNAs. In the RNaseH dependent mechanism, the double stranded mRNA/antisense RNA that is formed is degraded by RNaseH.

As used herein, “decrease” with respect to expression from a selected nucleic acid molecule refers to a decrease in expression in a detectable and statistically significant amount. For example, a decrease can refer to a 5%, 10%, 25%, 50%, 75%, or more than 75% decrease in expression. A decrease in expression also includes complete inhibition of expression, whereby greater than 95% decrease in expression from a nucleic acid molecule is achieved. Expression can be assessed by examining RNA levels, polypeptide levels, or phenotype.

A decrease in expression from a selected nucleic acid molecule can be achieved using one modified polynucleotide. A decrease of expression from a selected nucleic acid molecule also can be achieved using two modified polynucleotides having different sequences and complementary to different portions of the same selected nucleic acid molecule. Modified polynucleotides also can be used to decrease expression from more than one selected nucleic acid molecule. For example, multiple morpholino-modified polynucleotides having sequences complementary to more than one selected nucleic acid molecule can be used simultaneously to decrease expression from more than one selected nucleic acid molecule.

Modified polynucleotides such as morpholino-modified polynucleotides can be delivered to a living cell, tissue, organ, or organism of interest by methods used to deliver single stranded mRNA such as those described in (Hyatt and Ekker (1999) Methods in Cell biology 59:117-126). Examples of delivery methods include (1) microinjection and (2) simply exposing the cell, tissue, organ, or organism of interest to the polynucleotide analogue. Modified polynucleotides can be delivered in a suitable buffer. A suitable buffer is one in which the modified polynucleotide can be dissolved and that is non-toxic to the cell, tissue, organ, or organism to which the modified polynucleotide is to be delivered. A non-toxic buffer can be one that is isotonic to the organism or cell of interest. For example, morpholino-modified polynucleotides can be dissolved in Danieu buffer for injection into zebrafish eggs or embryos. A cell can be a fertilized or unfertilized egg, or a cell in culture. A tissue can be any tissue regardless of its state of differentiation, and can include, for example, tumors and joints. An organ can be thymus, bone marrow, pancreas, heart, or the blood vessels of the vasculature. An organism can be a vertebrate embryo such as a teleost embryo, a juvenile animal, or an adult animal. Examples of teleost embryos include zebrafish embryos, puffer fish embryos, medaka embryos, and stickleback embryos.

3. Inhibition of Expression by Unmodified Polynucleotides

In addition to modified polynucleotides, an unmodified polynucleotide (i.e., polynucleotide) having a sequence that is complementary to a selected nucleic acid molecule can be used to decrease expression from the selected nucleic acid molecule in a cell of interest. For example, a polynucleotide having a sequence that is antisense to an mRNA molecule can interfere with translation of the mRNA molecule. A polynucleotide having a sequence that is complementary to the DNA molecule from which an mRNA is transcribed, i.e. an antigene, also can interfere with transcription of the DNA molecule.

To decrease expression from a nucleic acid molecule, an antisense or antigene polynucleotide can be delivered into the cell of interest by any known method used to introduce nucleic acids into a cell. Alternatively, the antisense or antigene can be inserted into an expression vector that is then introduced into the cell of interest. In this case, the antisense or antigene polynucleotide is operably linked to an expression control sequence that directs the production of additional antisense or antigene polynucleotides. As used herein, the term “expression control sequence” refers to a nucleic acid sequence that modulates expression of a coding nucleic acid sequence. An expression control sequence can include, without limitation, a promoter, transcriptional enhancer elements, and any other nucleic acid elements required for RNA polymerase binding, initiation, and termination of transcription. As used herein, the term “operably linked” refers to covalent linkage of an expression control sequence and one or more coding nucleic acid sequences in such a way as to permit or facilitate expression of the coding nucleic acid sequences. Thus, the coding nucleic acid sequence can encode an antisense or antigene polynucleotide that is capably of annealing with a selected nucleic acid leading to alteration in expression from the selected nucleic acid in a sequence-specific manner. Therefore, antisense or antigene polynucleotides include heterologous (e.g. chemically synthesized) antisense or antigene polynucleotides that are introduced into a cell, as well as antisense or antigene polynucleotides produced within a cell.

4. HSST Polynucleotides

HSST polynucleotides include modified polynucleotides or unmodified polynucleotides that can alter expression from HSST nucleic acids in a sequence-specific manner by annealing with the HSST nucleic acids under intracellular conditions.

As used herein, the term “HSST nucleic acids” includes nucleic acids that are involved in the expression of polypeptides having heparan sulfate 6-O-sulfotransferase activities, i.e., HSST polypeptides. HSST nucleic acids can have HSST coding sequences as well as HSST noncoding sequences. HSST coding sequences include sequences encoding fragments of, or full-length, HSST polypeptides, while HSST non-coding sequences include, without limitation, untranslated sequences upstream of the translational start codon of an HSST coding sequence, as well as sequences upstream of a transcriptional start site of an HSST coding sequence. Examples of HSST nucleic acids are provided in GenBank accession numbers AI959303 and AK027720.

HSST polynucleotides can have sequences that are complementary to (1) noncoding regions, (2) coding regions, or (3) noncoding and coding regions of HSST nucleic acids. Examples of modified HSST polynucleotides that are complementary to the 5′ untranslated region of a selected nucleic acid include ec1-MO #1 (SEQ ID NO: 12) and ec1-MO#2 (SEQ ID NO: 13), which are complementary to the 5′ untranslated region of the zebrafish HSST nucleic acid sequence shown in FIG. 1.

5. HSST Polypeptides and HSST Antibody

HSST polypeptides can be identified by an activity assays, see for example Toyoda et al. (2000) J of Biol Chem 275:21856-21861. Polypeptides classified as belonging to the HSST family of polypeptides typically have two putative 3′-phosphoadenosine 5′-phosphosulfate (PAPS) binding sites, see Habuchi et al. (2000) J Biol Chem 275:2859-2868. Examples of polypeptides belonging to the HSST family of polypeptides include three mouse HSST homologues described in Habuchi et al. (2000) J Biol Chem 275:2859-2868. Another example of a polypeptide belonging to the HSST family of polypeptides include the human HS6ST described in Habuchi et al. (1998) J Biol Chem 273:9208-9213. A new polypeptide can be identified as belonging to the HSST family of polypeptides by amino acid or nucleic acid sequence comparison with known HSST polypeptides. For example, a newly identified polypeptide can be classified as belonging to the HSST family of polypeptide if the newly identified polypeptide is more similar to any member of the HSST family of polypeptides than the two least similar members within the HSST family. Methods for comparison of amino acids or nucleic acid sequences are known in the art and include BLAST analysis. Alternatively, a newly identified polypeptide can be classified as belonging to the HSST family of polypeptides if the newly identified polypeptide has the characteristic conserved domains described above. A newly identified polypeptide can be classified as belonging to the HSST family of polypeptides if the newly identified polypeptide has HSST activity as determined by known methods (see, for example, Toyoda et al. (2000) J Biol Chem 275:21856-21861). As used herein, the term “HSST polypeptide” refers to a polypeptide belonging to the HSST family of polypeptides.

A newly identified HSST polypeptide, or an immunogenic fragment of a newly identified HSST polypeptide, can be used to generate a specific antibody. As used herein, the term “polypeptide” includes both a full-length polypeptide and an immunogenic fragment of the full-length polypeptide. An immunogenic fragment refers to a polypeptide fragment that does not elicit an antibody response that is cross-reactive with another polypeptide. For example, an immunogenic fragment of one HSST polypeptide does not elicit an antibody response that is cross-reactive with another HSST polypeptide. A specific antibody directed towards one HSST polypeptide is one that is not cross-reactive with any other polypeptide including another HSST polypeptide. For example, a specific antibody directed to a newly identified HSST polypeptide will bind or hybridize specifically with the newly identified HSST polypeptide without substantially binding or hybridizing to other polypeptides that may be present in the same biological sample.

The term “antibody” as used herein refers to monoclonal, polyclonal, and recombinant antibodies as well as immunologically active fragments of antibodies. A monoclonal antibody is a homogenous population of antibody molecules. All antibody molecules of the monoclonal antibody population have the same antigen-binding site and bind the same epitope on an antigen. In contrast, a polyclonal antibody is a heterogeneous population of antibody molecules. Antibody molecules of the polyclonal antibody population recognize different epitopes of the same antigen. A recombinant antibody is a non-naturally occurring antibody that is encoded by a recombinant nucleic acid molecule. Typically, a non-naturally occurring antibody has portions that come from different organisms or different sources. One example of a non-naturally occurring antibody is a chimeric humanized antibody that consists of a human portion and a non-human portion. An immunologically active fragment of an antibody has the same antigen-binding site and therefore the same antigen specificity as the whole antibody. Examples of immunologically active fragments include F(ab) and F(ab′)2 fragments.

Monoclonal or polyclonal antibody can be produced using various methods. One method involves immunizing a non-human host animal with purified polypeptide antigen. The non-human host animal also can be immunized with a recombinant nucleic acid molecule that has a coding region for the antigen. See Chowdhury et al. (2001) J Immunol Methods 249:147-154 and Boyle et al. (1997) Proc Natl Acad Sci U.S.A 94:14626-31. This recombinant nucleic acid molecule also has an expression control sequence operably linked to the antigen-coding region and allow for antigen expression.

The non-human host animal that is immunized for antibody production can be, without limitation, a rabbit, a chicken, a mouse, a guinea pig, a rat, a sheep, or a goat. Blood serum from the immunized non-human host animal is used as a source of polyclonal antibody. To obtain a polyclonal antibody from the blood serum of an immunized host animal, any standard method can be used. Typically, the polyclonal antibody is obtained from blood serum using protein A chromatography.

A monoclonal antibody can be obtained using B-lymphocytes isolated from an immunized non-human host animal. Typically, antibody-producing B-lymphocytes are isolated from the spleen of the immunized host animal at a time after immunization when serum antibody titer is highest. Serum antibody titer can be determined using any standard method. For example, enzyme linked immunosorbent assay (ELISA) can be used to determine the titer of an anti-CRISP-3 antibody in a sample. In ELISA, the antigen typically is immobilized on a surface. The immobilized antigen is exposed to serum containing the specific antibody under conditions that allow for specific binding of the antibody to the antigen. The bound antibody can be detected with a second antibody that is conjugated with a readily detectable marker such as an enzyme, a fluorescent molecule, or a radioactive molecule. Once isolated, B-lymphocytes are fused with myeloma cells to generate hybridoma cells. Standard hybridoma fusion methods are described in Kohler and Milstein (1975) Nature 256:495-497 and Kozbor et al. (1983) Immunol Today 4:72. Hybridoma cells are cultured singly so that each culture results from the growth of one hybridoma cell. An antibody-producing hybridoma cell can be identified by screening culture supernatants of different hybridoma cell cultures for an antibody that binds to the antigen of interest. The antibody in the supernatant can be identified using ELISA as described above.

A monoclonal antibody also can be obtained by using commercially available kits that aid in preparing and screening antibody phage display libraries. An antibody phage display library is a library of recombinant combinatorial immunoglobulin molecules. Examples of kits that can be used to prepare and screen antibody phage display libraries include the Recombinant Phage Antibody System (Pharmacia) and SurfZAP Phage Display Kit (Stratagene).

A recombinant chimeric humanized antibody, an immunologically active immunoglobulin fragment, and a single chain antibody specific for a particular polypeptide antigen can be prepared using known techniques such as those described in Better et al. (1988) Science 240:1041-1043, Jones et al. (1986) Nature 321:552-525, and U.S. Pat. Nos. 4,946,778 and 4,704,692. A chimeric humanized antibody can be produced by combining a portion of a mouse antibody coding sequence specific for the antigen of interest with a portion of a human antibody coding sequence. An immunologically active immunoglobulin fragment such as a F(ab′)2 fragment can be generated by digestion of an antibody with pepsin while a F(ab) fragment can be obtained by reduction of the disulfide bridges of the F(ab′)2 antibody fragment. A single chain antibody can be formed by linking the heavy and light chains of an immunoglobulin molecule together with an amino acid bridge.

6. HSST-Modulating Agents

The invention provides a method for identifying a substance that decreases or increases the amount or activity level of an HSST polypeptide in a cell. A substance that decreases or increases the amount or activity level of an HSST polypeptide is herein referred to as an “HSST-modulating agent.” The amount or activity level of HSST polypeptide can be assessed by determining HSST enzymatic activity using known methods, by detecting HSST polypeptide using antibody-based assays, or by detecting HSST RNA using nucleic acid-based assays. The amount of HSST polypeptide in a cell can be decreased or increased by modulating HSST polypeptide expression. HSST polypeptide expression can be modulated by decreasing or increasing the production of functional HSST mRNA or the amount of functional HSST polypeptide.

The invention also provides a method for identifying a substance that decreases or increases the enzymatic activity of an HSST polypeptide in a purified or partially purified HSST polypeptide preparation. A substance that decreases or increases HSST enzymatic activity is also herein referred to as an “HSST-modulating agent.” The activity of HSST can be determined using methods known in the art. See, for example, Toyoda et al. (2000) J Biol Chem 275:21856-21861.

As used herein, the term “purified or partially purified,” with respect to a polypeptide preparation, describes a composition containing the polypeptide of interest at a stage subsequent to initiation of a polypeptide purification procedure. Typically, a polypeptide purification procedure consists of combinations of one or more of, for example, centrifugation or filtration of cell culture media or cell/tissue lysates, polypeptide precipitation, filtration, and chromatographic separation steps designed to enrich for the polypeptide of interest and remove unnecessary components. In a partially purified polypeptide preparation, the polypeptide of interest is enriched by some amount, for example 2.5%, 5%, 10%, 20% 50% or greater than 50% by weight compared to the amount of the polypeptide of interest present prior to initiation of the purification procedure. In a partially purified polypeptide preparation, the polypeptide of interest is not purified to homogeneity, however, and would not appear as a single band upon gel electrophoresis. In a purified polypeptide preparation, the polypeptide of interest typically accounts for greater than 90% by weight of the entire content of the preparation, and may appear as a single band upon gel electrophoresis.

To identify HSST-modulating agents, a cell producing HSST polypeptides or a purified or partially purified HSST polypeptide preparation can be contacted with a test agent. The amount or activity of the HSST polypeptide in the cell or partially purified HSST polypeptide preparation is determined. The HSST-modulating agent is one that causes an increase or decrease in the amount or activity level of HSST polypeptides relative to a control cell or a control HSST polypeptide preparation. A control, with reference to a cell or polypeptide preparation, can be, for example, a preparation that has not been contacted with a test agent.

As used herein, the term “decrease” or “increase” refers to a detectable change, for example, a 3%, 6%, 12%, or greater than 12% decrease or increase, that is statistically significant.

7. Angiogenesis-Modulating Agents

Angiogenesis refers to generation of new blood vessels. Under normal physiological conditions, angiogenesis occurs under particular conditions such as in wound healing, during tissue and organ regeneration, during embryonic vasculature development, as well as in the formation of the corpus luteum, endometrium, and placenta. Excessive angiogenesis, however, has been associated with a number of disease conditions. Examples of diseases associated with excessive angiogenesis include rheumatoid arthritis, atherosclerosis, diabetes mellitus, retinopathies, psoriasis, and retrolental fibroplasia. In addition, angiogenesis has been identified as a critical requirement for solid tumor growth and cancer metastasis. Examples of tumor types associated with angiogensis include rhabdomyosarcomas, retinoblastoma, Ewing's sarcoma, neuroblastoma, osteosarcoma, hemangioma, leukemias, and neoplastic diseases of the bone marrow involving excessive proliferation of white blood cells. Due to the association between angiogenesis and various disease conditions, substances that have the ability to modulate angiogenesis would be potentially useful treatments for these disease conditions.

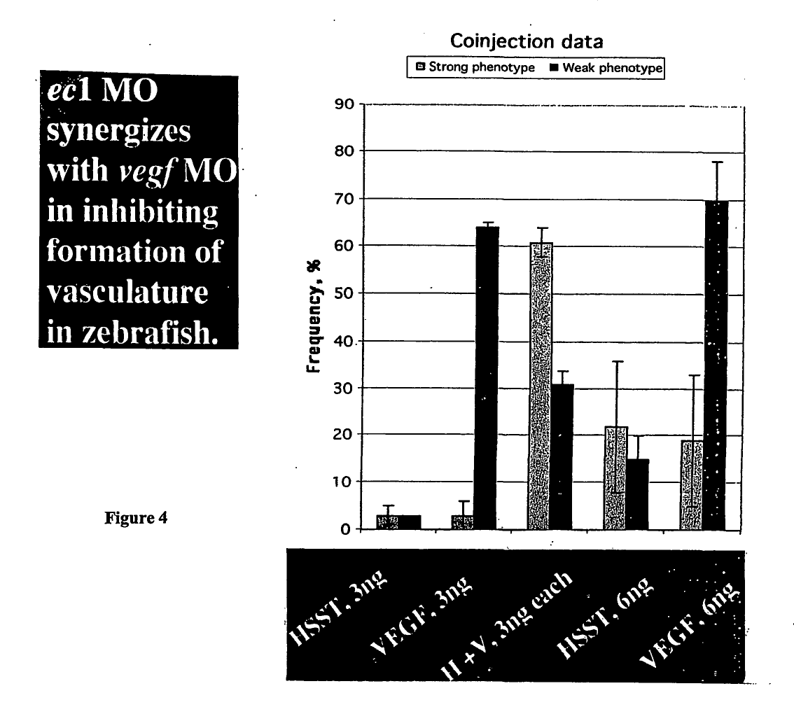

The invention provides a method for modulating angiogenesis in an animal. The method involves introducing an HSST modulating agent into the animal in an amount effective to modulate angiogenesis. As used herein, the phrase “effective amount” or “amount effective to” refers to an amount of a substance that is required to achieve a particular phenotype. For example, an effective amount of a morpholino-modified polynucleotide such as ec1-MO (SEQ ID NO: 12) for mediating the strong or weak phenotype associated with vasculature formation in zebrafish is 6 ng per embryo, while an effective amount of the vegf-MO (SEQ ID NO: 15) for achieving similar phenotypes in zebrafish is 3 ng per embryo (see Example 13 and FIG. 4).

As used herein, an animal includes a vertebrate animal such as a fish, a mouse, a rabbit, a guinea pig, a pig, and a monkey. The animal can be an embryo, a juvenile animal, or an adult. The animal also can be a human.

The invention also provides a method for identifying a substance that (1) is an HSST-modulating agent and that (2) alters the typical pattern, course, or extent of angiogenesis in a healthy or diseased tissue, organ, or organism. An HSST-modulating agent that also alters the typical pattern, course, or extent of angiogenesis is herein referred to as an “angiogenesis-modulating agent.” An HSST-modulating agent can decrease angiogenesis in a localized tissue or organ, for example in a solid tumor. An HSST-modulating agent also can decrease angiogenesis in a systemic fashion and in some cases, to the extent that no vasculature development occurs. For example, a developing zebrafish embryo exposed to an HSST-modulating agent may be devoid of vasculature. To identify angiogenesis-modulating agents, an animal can be contacted with an HSST-modulating agent. The animal is then monitored for any alteration in angiogenesis. The angiogenesis-modulating agent is one that causes any alteration in angiogenesis in the animal.

The invention will be further described in the following examples, which do not limit the scope of the invention described in the claims.

### EXAMPLES

### Example 1

### Zebrafish Care and Egg Collection

Standard zebrafish care protocols are described in Westerfield (1995) Oregon: University of Oregon Press.

Zebrafish were kept in 6.5-gallon (26 liters) and 20-gallon (76 liters) plastic tanks at 28° C. A 6.5-gallon tank housed 25 fish and a 20-gallon tank housed 70 fish. Tank water was constantly changed with carbon-filtered and UV-sterilized tap water (system water) at a rate of 15 to 40 nL/min or was replaced each day by siphoning up debris from the bottom of the tank. Tap water, aged a day or more in an open (heated) tank to release chlorine, was adequate, although more consistent conditions were obtained by adding commercial sea salts to deionized or distilled water (60 mg of ‘Instant Ocean’ salt per liter of water, see Westerfield (1995) Oregon: University of Oregon Press.). A 10-hour dark and 14-hour light day cycle was maintained in zebrafish facility.

Fish were fed brine shrimp twice a day. To make shrimp, 100 mL of brine shrimp eggs were added to 18 L of salt water (400 mL of ‘Instant Ocean’ salt per 18 L of water) and aerated vigorously. After 2 days at 28° C., the shrimp were filtered through a fine net, washed with system water, suspended in system water, and fed to fish. Alternatively, fish could also be fed with ‘Tetra’ brand dry flake food.

Zebrafish spawning was induced every morning shortly after sunrise. To collect the eggs, a ‘false bottom container’ system was used (Westerfield (1995) Oregon: University of Oregon Press). The system consisted of two containers of approximately 1.5 L, one slightly smaller than the other. The bottom of the smaller container was replaced with a stainless steel mesh with holes bigger than the diameter of zebrafish eggs. The smaller container was then placed into the bigger container, and the setup was filled with system water. Up to eight zebrafish were placed inside the smaller container. When the fish spawn, the eggs fall through the mesh into the bigger container, and in this way, the eggs cannot be reached by the fish and eaten. About 10-15 minutes were allowed for spawning, after which time the smaller container with the fish was transferred into another bigger container. The eggs were collected by filtration using a mesh with the holes smaller than the diameter of the eggs. Fish were used once a week for optimal embryo production.

### Example 2

### Identification of a Zebrafish Gene, ec1, Encoding an HSST Homologue

Using blast analysis, a clone containing a coding sequence with strong similarity to mouse heparan sulfate 6-sulfotransferase (HS6ST) 2 or 3 was identified in a zebrafish EST database. The partial sequence (accession number AI959303) reported in the zebrafish EST database includes about 230 nucleotides of the 5′ untranslated region and about 500 nucleotides of the coding sequence. The coding sequence, corresponding to a zebrafish HS6ST gene, was named zebrafish ec1.

To obtain the full-length zebrafish ec1 coding sequence, automatic sequencing reactions were performed using primers designed from the partial sequence reported in the database. The following primers were used to obtain the complete sequence of the ec1 open reading frame:

| | |
|---|---|
| Vector primer m13r—: | |
| 5′tcctgtgtgaaattgttatcc 3′ | (SEQ ID NO: 5) |
| 5′ctcctcagtacacatatgg 3′: | (SEQ ID NO: 6) |
| 5′gctgatgggacagtggatt 3′: | (SEQ ID NO: 7) |
| 5′gaagaagtgtacctgctac 3′ | (SEQ ID NO: 8) |
| 5′cacggtaatgagccgagaa 3′ | (SEQ ID NO: 9) |
| 5′ccagcgtttgttcagcatc 3′ | (SEQ ID NO: 10) |
| 5′ctggctgttctcccgctt 3′ | (SEQ ID NO: 11) |

The full-length sequence of the zebrafish ec1 gene is shown in FIG. 1 (SEQ ID NO: 1). The sequence for part of the 5′ untranslated region is shown in lower case. The polypeptide sequence of zebrafish EC1 (SEQ ID NO. 2) showed 60% sequence identity with mouse HS6ST-2 and 72% sequence identity to mouse HS6ST-3.

### Example 3

### Spatial Expression Pattern of Zebrafish ec1 in Early Zebrafish Embryos

To determine the expression pattern of ec1 throughout the early development of zebrafish embryos, in situ hybridizations were performed. The zebrafish eclgene was labeled with digoxigenin and used as a probe. In situ hybridization was performed as described in Jowett et al. (1999) Methods in Cell Biology 59:63-85.

The spatial expression pattern of ec1 was determined at different embryonic stages. At the 4-somite stage (11.5 hours post-fertilization), ec1 was expressed along the somitic mesoderm. At the 26-somite stage (22 hours post-fertilization), ec1 was expressed in a cluster of cells ventrolateral to the notochord in the tail region. At 24 hours post-fertilization, ec1 was expressed in three sets of bilateral patches: one set by the yolk and near the head, a second set in the upper trunk, and the third set in the tail region.

In addition, during somitogenesis, ec1 expression was progressively confined to maturing somites in the posterior region of the embryo, specifically in the anterior half of each somite. By the 20-somite stage, expression of ec1 was detected only in the posterior 4-5 somites. Expression of ec1 in the somitic mesoderm disappeared by the 26-somite stage (22 hours post-fertilization).

### Example 4

### Spatial Expression Pattern of ec1 Overlaps with Spatial Expression Pattern of SCL in Early Zebrafish Embryos

The spatial expression pattern of ec1 was compared, using in situ hybridization, with that of SCL, a transcription factor expressed in both endothelial and hematopoietic precursor cells (Gering et al. (1998) EMBO J. 17:4029-4045). The ec1 hybridization probe used was the zebrafish encoding sequence labeled with digoxigenin. The SCL hybridization probe, also labeled with digoxigenin, was the scl coding sequence described in Gering et al. (1998) EMBO J 17:4029-4045. In situ hybridization was preformed as described in Example 3.

At 22 hours, the spatial expression pattern of ec1 overlapped with that of SCL at the same stage. Therefore, it is possible that ec1-expressing cells in the tail regions of zebrafish embryos give rise to both endothelial cells that line the blood vessels as well as red and white blood cells.

### Example 5

### Morpholino Inactivation of Zebrafish ec1

To determine the function of ec1 in early zebrafish development, morpholino-modified polynucleotides (MOs) that target the 5′ untranslated region of zebrafish ec1 were made and used for decreasing ec1 expression. The two zebrafish ec1-MOs had the following sequences:

| | |
|---|---|
| Ec1-MO #1: | |
| 5′GATTTCCCATCCATCTTCTCGCTGG 3′ | (SEQ ID NO: 12) |
| Ec1-MO #2: | |
| 5′AGTGAAAGCATTACTCGGTTGTGCG 3′ | (SEQ ID NO: 13) |

In addition, an ec1-MO with a 4-base mismatch, designated ec1-MO (Δ4), was used to assess the specificity of ec1-MO targeting. Ecl-MO (Δ4) had the following sequence: 5′agtCaaTgcattaGtcggttCtgcg 3′ (SEQ ID NO: 14). The mismatched bases are indicated by capital letters.

Morpholino phosphorodiamidates (morpholinos) were obtained from Gene Tools, LLC and were designed to bind to the 5′ untranslated regions including the initiating methionine. Sequences were design based on parameters recommended by the manufacturer. For example, 21-25 base polynucleotides of approximately 50% G/C and 50% A/T content were generated. Internal hairpins as well as four consecutive G nucleotides were avoided.

Morpholino-modified polynucleotides were solubilized in water at the concentration of 8 mM (approximately 65 mg/nL) or 50 mg/mL. The resulting stock solution was diluted to worldng concentrations of 0.09 to 3 mg/mL in water or 1×Danieau solution. Danieau buffer consisted of 8 mM NaCl, 0.7 mM KCl, 0.4 mM MgSO₄, 0.6 mM Ca(NO₃)₂, and 5.0 mM HEPES (pH 7.6). Zebrafish embryos at the 1 to 4 cell stages were microinjected with 4-9 nL of morpholino-modified polynucleotides.

Morpholino injection method was very similar to the mRNA injection method described in Hyatt and Eldcer (1999) Methods in Cell Biology 59:117-126. The collected eggs were transferred onto agarose plates as described in Westerfield (1995) Oregon: University of Oregon Press. While agarose plates for mRNA injections were kept cold to slow embryo development, the plates for morpholino injections were prewarmed to approximately 20° C., since morpholino injection into cold embryos were found to increase non-specific effects and mortality of the injected embryos.

Needles used for morpholino injections were the same as for mRNA injections (Hyatt and Eklcer (1999) Methods in Cell Biology 59:117-126). The needles were back-filled with a pipette and calibrated by injecting the loaded morpholino solution into a glass capillary tube. The picoinjector volume control was then setup for 1.5 to 15 nL. The injection volume depended on the required dose, usually 1.5 to 18 ng of morpholino was injected. Morpholino solutions were injected through the chorion into the yolk of zebrafish embryos. The injected embryos were transferred to petri dishes containing system water and allowed to develop at 28° C.

### Example 6

### Morphology of Zebrafish Embryos Injected with ec1-MO

The phenotypes of zebrafish embryos injected with morpholino-modified polynucleotides were first assessed by visual inspection using dissecting microscopes. Microscopic observations showed that the overall morphology of embryos injected with ec1-MO was relatively normal up to about 28 hours post-fertilization.

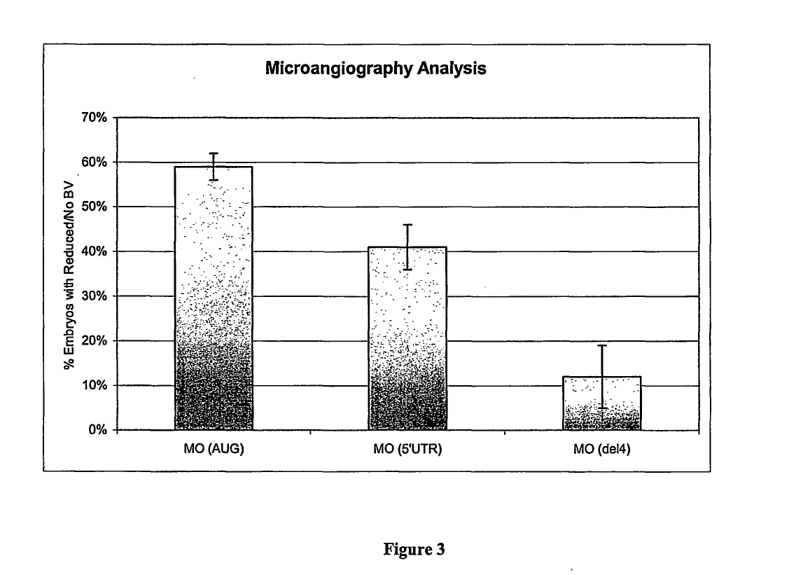

At about 28 hours post-fertilization, however, embryos began to exhibit enlarged pericardial sacs and showed lack of circulation. By 48 hours, the enlarged pericardial sac became more apparent, and pericardial edema was observed. Blood accumulation was seen underneath the yolk, adjacent to the pericardial sac and occasionally in the tail. This effect was specific as injection of either ec1-MO #1 or ec1-MO #2 gave rise to the same phenotype. Moreover, injection with a comparable dose of the 4-base mismatched morpholino-modified polynucleotide, ec1-MO (Δ4), produced a significantly lower percentage of embryos exhibiting the above phenotype. (See FIG. 3.)

### Example 7

### Microangiogragph Analysis of Zebrafish Embryos Injected with ec1-MO

To determine whether the vasculature in zebrafish embryos injected with ecl-MO formed properly, microangiography was performed on both uninjected control embryos and embryos injected with ec1-MO. In microangiography, fluorescent FITC-Dextran dye is microinjected into the common cardinal vein of zebrafish embryos as described in Nasevicius et al. (2000) Yeast 17:294-301. Between 10-15 nL of FITC-Dextran fluorescent dye (1 μg/mL) was microinjected into 48-hour embryos incubating in 0.004% Tricain solution. The dye is taken to the heart and then pumped into the systemic circulation, allowing visualization of the entire vasculature using fluorescent microscopy. Results of microangiography showed that embryos injected with ec1-MO exhibited defects in vasculogenesis (initial formation of axial vessels) and angiogenesis (sprouting of new vessels from existing axial vessels).

FIG. 3 shows the percentages of embryos exhibiting decreased or no blood vessel formation subsequent to injection with ec1-MO #1, ec1-MO #2, or the control ec1-MO (Δ4).

### Example 8

### Initial Specification of Red Blood Cell Fate was Normal in Embryos Injected with ec1-MO

To determine whether lack of circulation observed in zebrafish embryos injected with ec1-MO resulted from insufficient red blood cell production, the initial specification of red blood cells (RBCs) was examined. Progenitor cells that give rise to RBCs express the transcription factor GATAL. The expression of GATAL was compared in wild-type zebrafish embryos and in embryos injected with ec1-MO using in situ hybridization. The GATAL probe, also labeled with digoxigenin, was the gatal gene described in Detrich et al. (1995) Proc Natl Acad Sci USA 92:10713-10717. Results showed that expression of GATAL was relatively normal in zebrafish embryos injected with ec1-MO, suggesting that the initial specification of RBC fate is normal in zebrafish embryos injected with ec1-MO.

Initial production of RBCs in zebrafish embryos also was examined by microinjection of a morpholino-modified polynucleotide targeted to a gene encoding uroporphyrinogen decarboxylase. Uroporphyrinogen decarboxylase (UROD) is an enzyme involved in the biosynthesis of heme in RBCs. A decrease in expression of urod by morpholino-modified polynucleotide targeting results in the inactivation of the enzyme and subsequent accumulation of fluorescent RBCs. The urod-MO used is described in Nasevicius and Ekker (2000) Nature Genetics 26:216-220. Urod-MO preparation and injection were performed as described for ec1-MO. Fluorescent RBCs were observed in both wild-type zebrafish embryos and embryos injected with ec1-MO at about 28 hours post-fertilization upon injection with urod-MO. Therefore, initial production of RBCs was normal in embryos injected with ec1-MO.

### Example 9

### Initial Specification of Endothelial Cell Fate was Normal in ec1-MO Injected Embryos

To determine whether vascular defects observed in embryos injected with ec1-MO resulted from defects in initial specification of endothelial cell fate, the expression of both early and late vascular markers was examined using in situ hybridization. Fli1 and flk1 are vascular markers expressed in endothelial cells early in vascular development. Expression of fli1 and flk1 was examined in uninjected control embryos and embryos injected with ec1-MO. Results showed that expression of these two markers was relatively normal in embryos injected with ec1-MO.

In addition, late differentiation of endothelial cells also appeared normal in zebrafish embryos injected with ec1-MO as expression of late vascular markers, tie1 and tie2, was found to be normal.

### Example 10

### Normal Formation of Notochord and Hypochord in Embryos Injected with ec1-MO

To determine whether formation of the notochord and hypochord was normal in embryos injected with ec1-MO, expression of no tail (ntl) and cs1 was analyzed in both uninjected embryos and embryos injected with ec1-MO. In situ hybridization was performed using digoxigenin-labeled ntl (see Schulte-Merker et al. (1992) Development 116:1021-1032) and cs1 (Saunders, C., Larson, J. D., and Ekker, S. C., unpublished data) probes. Results showed that expression of both ntl and cs1 was relatively normal in embryos injected with ec1-MO. This result suggested that formation of the notochord and hypochord was normal. Therefore, vasculature defects did not result from formation of defective midline structures.

### Example 11

### Zebrafish Embryos Injected with ec1-MO Exhibit Altered Somitic Expression of ptc-1

While the formation of midline structures such as the notochord and the hypochord was normal in embryos injected with ec1-MO, it is possible that the response to signals generated from the midline structures is abnormal. For example, Sonic hedgehog (SHH) produced by the notochord has been implicated in the formation of axial vessels in zebrafish. A sonic hedgehog mutant, sonic you (syu), exhibited no obvious defects in the notochord and the hypochord formation. The mutant had a single axial vein and lacked the dorsal aorta (see Roman and Weinstein (2000) BioEssays 22: 882-893). The signaling pathway mediated by SHH eventually leads to induction of several genes including patched-1 (ptc-1). Therefore, if the response to midline signals such as SHH is lacking in embryos injected with ec1-MO, then the expression of patched-1 would be reduced in these embryos. To address this possibility, in situ hybridization experiments were performed to assess expression of ptc-1 in both uninjected embryos and embryos injected with ec1-MO. Results showed that in wild-type embryos, ptc-1 was expressed in the presumptive adaxial and somitic mesoderm adjacent to the notochord. In contrast, embryos injected with ec1-MO expressed ptc-1 in adaxial cells along the anterior-to-posterior axis. Disorganized expression of ptc-1 was observed in the somitic mesodermal cells. Results from two in situ experiments indicated that when 6 ng of ec1-MO #1 were injected into embryos, 66% (+/−11%) of the injected embryos showed disorganized somitic ptc-1 staining. In addition, when 10 ng of ec1-MO #2 were injected into embryos, 33% (+/−1%) of injected embryos showed disorganized somitic ptc-1 staining. The expression of ptc-1 in ec-1-MO injected embryos indicates that the response to midline SHH signal is normal in these embryos. The disorganized expression of ptc-1 in somitic mesoderm observed in embryos injected with ec1-MO, however, is probably secondary to altered responses to other signaling pathways.

### Example 12

### Expression of Tie-2 in Embryos Injected with ec1-MO

Tie-2 encodes a tyrosine kinase receptor essential for late maturation and maintenance of vasculature (Puri et al. (1999) 126:4569-80). Expression of tie-2 in embryos injected with ec1-MO was examined using in situ hybridization. In embryos injected with ec1-MO, expression of tie-2 was unaffected at 33 hours post-fertilization. At 48 hours post-fertilization, however, expression of tie-2 was reduced. Progressive loss of tie-2 expression was also observed in angiopoetin-1 (ang-1) deficient mice (Suri et al., (1996) Cell: 1171-80). Ultrastructural analysis of blood vessels revealed defective formation of periendothelial cells and the collagen-like fibers in the surrounding matrix, suggesting the essential role of ang-1 in blood vessel stabilization. Loss of tie-2 expression at a later stage of vascular development in embryos injected with ec1-MO implicates EC1 in the maintenance of mature vasculature.

### Example 13

### Synergy of ec1-MO and vegf-MO

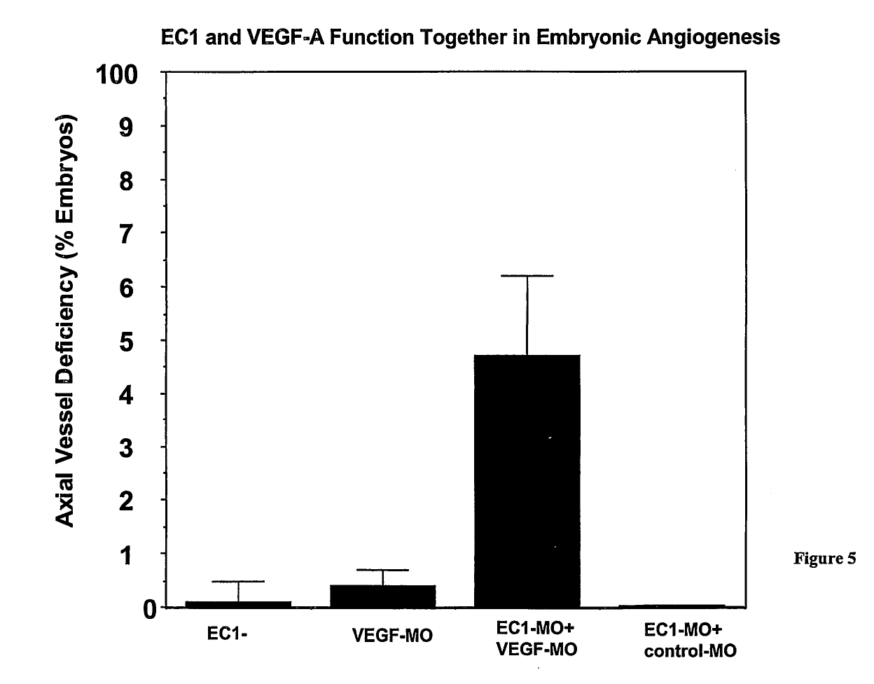

Signaling by members of the Vascular Endothelial Growth Factor (VEGF) gene family is implicated in the formation of vasculature during embryogenesis, during wound healing, and for the growth of tumor-induced vasculature (See Carmeliet et al. (1996) Nature 380:435-439; Carmeliet et al. (1997) Am J Physiol 273:H2091-104; and Ferrara (1999) J Mol Med 77:527-543). Since VEGF plays a central role in vasculogenesis and angiogenesis, effects due to decrease in expression from both ec1 and vegf were examined. Zebrafish embryos were injected with two MOs: an ec1-MO (5′GATTTCCCATCCATCTTCTCGCTGG 3′, SEQ ID NO: 12) and a vegf-MO (5′ GTATCAAATAAACAACCAAGTTCAT 3′, SEQ ID NO: 15). Morpholino injections and zebrafish phenotypic analysis were performed as described in Nasevicius et al. (2000) Yeast 17:294-301. FIG. 4 is a bar graph comparing the percentages of embryos exhibiting a strong or a weak VEGF morphant phenotype when injected with ec1-MO alone (HSST, 3 ng), vegf-MO alone (VEGF, 3 ng), ec1-MO and vegf-MO (H+V, 3 ng each), ec1-MO at 6 ng (HSST, 6 ng), or vegf-MO at 6 ng (VEGF, 6 ng). These results demonstrate that ec1-MO and vegf-MO together had a synergistic effect on inhibiting formation of vasculature in zebrafish. FIG. 5 is a bar graph comparing the percentages of embryos exhibiting axial vessel deficiency when injected with ecl-MO alone, the vegf-MO alone, both ec1-MO and vegf-MO, or ecl-MO and a control-MO. These results show that injections of both ec1-MO and vegf-MO had a synergistic effect on the percentages of embryos exhibiting defective axial vessels. Therefore, EC1 interacts either directly or indirectly with VEGF, and plays a role in angiogenesis.

### Example 14

### Identification of a Human HSST Gene from Human Fetal Liver cDNA Library

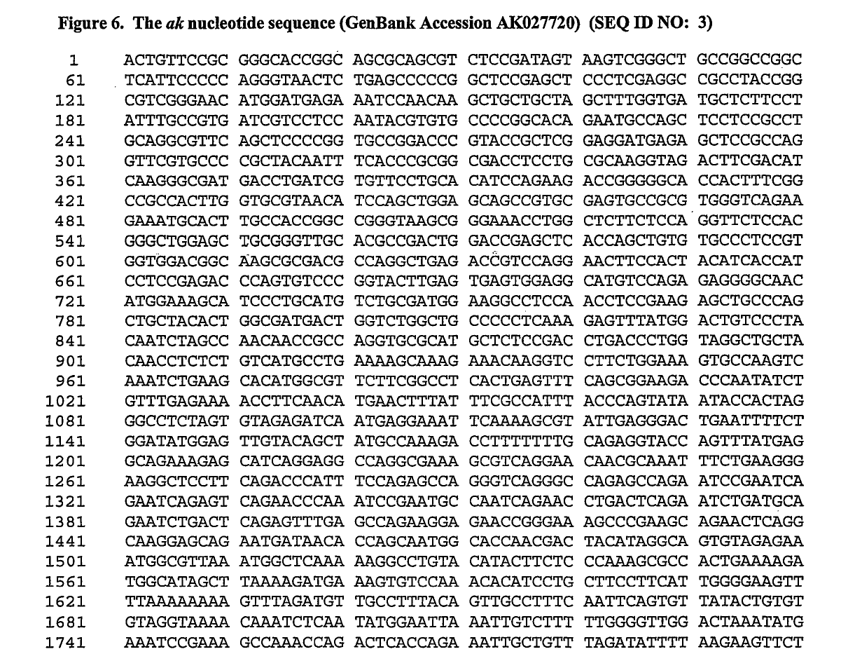

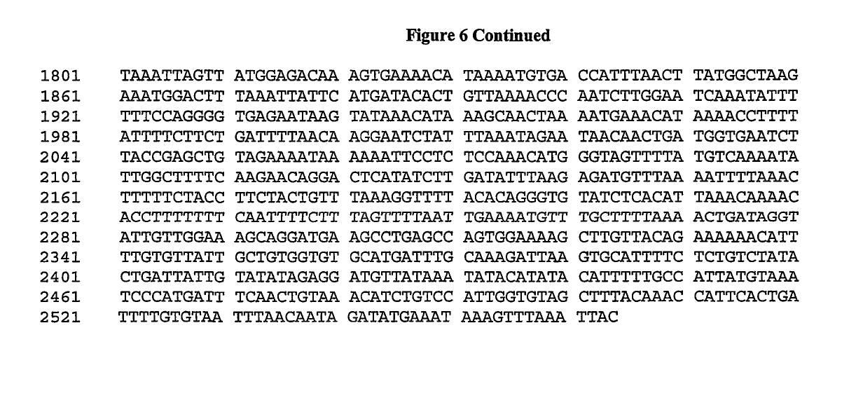

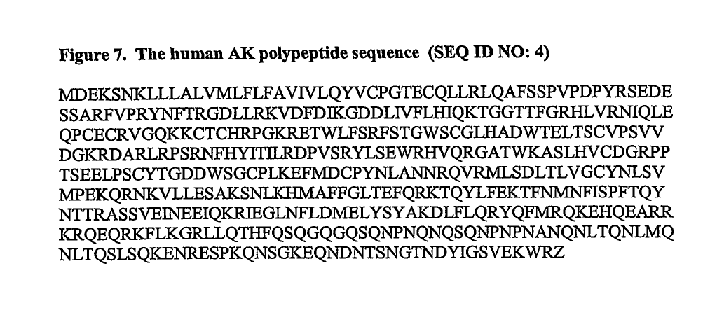

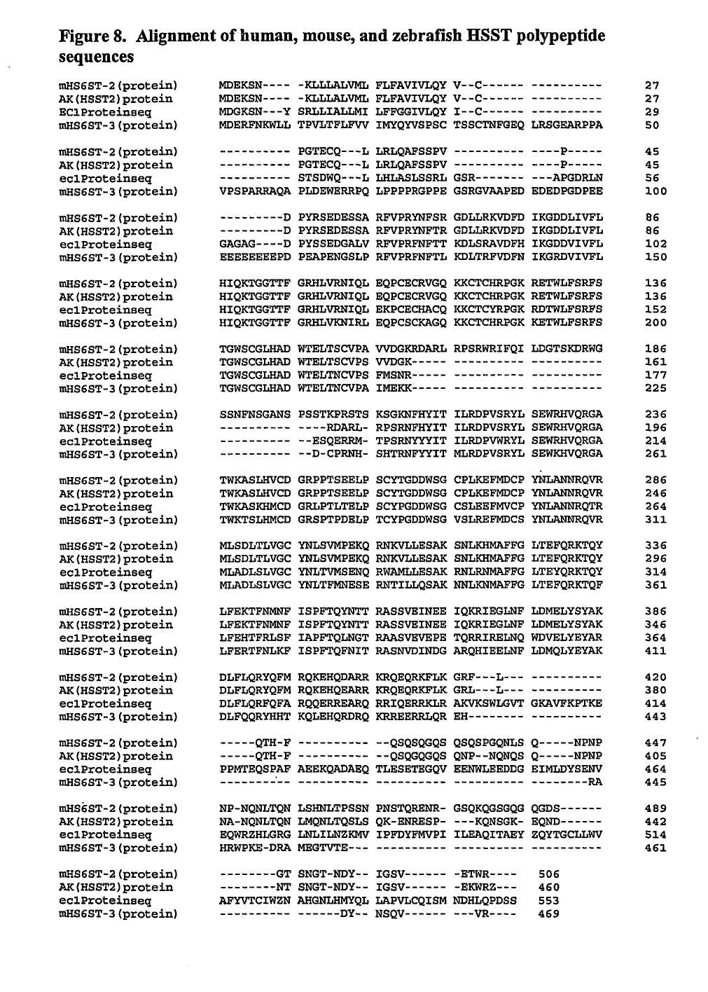

Using the zebrafish ec1 coding sequence, a human hsst gene, designated the ak isoform, was identified in a teratocarcinoma cell line (GenBank Accession AK027720). The complete ak coding sequence including 130 nucleotides of the 5′ untranslated region and 1054 nucleotides of the 3′ untranslated region is found in GenBank Accession AK027720 and shown in FIG. 6. The polypeptide sequence of the AK polypeptide (SEQ ID NO: 4) is shown in FIG. 7. The AK polypeptide is different from the polypeptide encoded by the HS6ST clone isolated from the human fetal brain cDNA library (Habuchi et al. (1998) J Biol Chem 273:9208-9213). FIG. 8 is an alignment of the AK polypeptide sequence with the zebrafish EC1 polypeptide sequence and two mouse HSST polypeptides.

To isolate an ak cDNA clone from a human fetal liver cDNA library, the ak coding sequence (SEQ ID NO: 3) was used to designed primers for PCR. The primer sequences used were:

| | |
|---|---|
| 5′GAAGATCTCACCATGGATG | (SEQ ID NO: 16) |
| AGAAATCCAACAAG 3′ and | |
| 5′GAAGATCTTTAACGCCATT | (SEQ ID NO: 17) |
| TCTCTACACT 3′. | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A morpholino-modified HSST polynucleotide, wherein said morpholino-modified HSST polynucleotide is complementary to a nucleic acid molecule that encodes an HSST polypeptide, and wherein said morpholino-modified HSST polynucleotide is effective to decrease expression from said nucleic acid molecule.

2. The morpholino-modified HSST polynucleotide of claim 1, wherein said HSST polypeptide has the sequence of SEQ ID NO: 2.

3. The morpholino-modified HSST polynucleotide of claim 1, wherein said HSST polypeptide has the sequence of SEQ ID NO: 4.

4. A cell comprising the morpholino-modified HSST polynucleotide of claim 1.

5. A teleost embryo comprising the morpholino-modified HSST polynucleotide of claim 1.

6. The teleost embryo of claim 5, wherein said teleost embryo is selected from the group consisting of a zebrafish embryo, a stickleback embryo, a medaka embryo, and a puffer fish embryo.

7. An expression vector comprising an expression control sequence and a coding sequence, wherein said expression control sequence directs production of a polynucleotide from said coding sequence, and wherein said polynucleotide is complementary to SEQ ID NO: 1 or 3.

8. A purified polypeptide comprising the amino acid sequence of SEQ ID NO: 2.

9. A purified antibody that binds specifically to a polypeptide comprising an amino acid sequence selected from the group consisting of SEQ ID NO: 2 and SEQ ID NO: 4.

10. A method of making an antibody comprising immunizing a non-human host animal with a polypeptide or an immunogenic fragment of said polypeptide, wherein said polypeptide has an amino acid sequence selected from the group consisting of SEQ ID NO: 2 and SEQ ID NO: 4.

11. A method of making an antibody, comprising providing a hybridoma cell that produces a monoclonal antibody specific for a polypeptide with an amino acid sequence selected from the group consisting of SEQ ID NO: 2 and SEQ ID NO: 4, and culturing the cell under conditions that permit production of the monoclonal antibody.

12. A method of identifying an HSST-modulating agent, said method comprising:
a) contacting a test agent with a cell producing an HSST polypeptide,
b) detecting the amount or activity level of said HSST polypeptide subsequent to step (a), and
c) identifying said test agent as an HSST-modulating agent if the amount or activity level of said HSST polypeptide is increased or decreased relative to a control cell.

13. A method of identifying an HSST-modulating agent, said method comprising:
a) contacting a test agent with a purified or partially purified polypeptide preparation comprising an HSST polypeptide,
b) detecting the activity of said HSST polypeptide subsequent to step (a), and
c) identifying said test agent as an HSST-modulating agent if said activity of said HSST polypeptide is increased or decreased compared to a control purified or partially purified HSST polypeptide preparation.

14. A method of identifying an angiogenesis-modulating agent, said method comprising:
a) contacting an animal with an HSST-modulating agent,
b) monitoring said animal for alteration in angiogenesis, and
c) identifying said HSST-modulating agent as an angiogenesis-modulating agent if alteration in angiogenesis is detected in step (b).

15. A method of making an angiogenesis-modulating agent, said method comprising:
a) contacting an animal with an HSST-modulating agent,
b) monitoring said animal for alteration in angiogenesis,
c) identifying said HSST-modulating agent as an angiogenesis-modulating agent if alteration in angiogenesis is detected in step (b), and
d) producing said angiogenesis-modulating agent.

16. A method of modulating angiogenesis in an animal, said method comprising introducing an HSST-modulating agent into said animal in an amount effective to modulate said angiogenesis.

17. The method of claim 16, wherein said HSST-modulating agent is selected from the group consisting of a polynucleotide and an antibody.

18. The method of claim 17, wherein said polynucleotide is a morpholino-modified polynucleotide.

19. The method of claim 17, wherein said polynucleotide is encoded by an expression vector.

20. The method of claim 16, wherein said animal is a zebrafish.
